(19) United States
(12) Patent Application Publication
(10) Pub. No.: US 2023/0270669 A1
(43) Pub. Date: Aug. 31, 2023

(54) PHARMACEUTICAL COMPOSITION

(71) Applicant: MEXICHEM FLUOR S.A. DE C.V., San Luis Potosi, S.L.P., C.P. (MX)

(72) Inventor: Stuart CORR, Warrington, Cheshire (GB)

(73) Assignee: MEXICHEM FLUOR S.A. DE C.V., San Luis Potosi, S.L.P., C.P. (MX)

(21) Appl. No.: 18/006,990

(22) PCT Filed: Sep. 2, 2021
(86) PCT No.: PCT/GB2021/052269

§ 371 Date: Jan. 26, 2023

Related U.S. Application Data
(60) Provisional application No. 63/074,814, filed on Sep. 4, 2020.

(51) Int. Cl. A61K 9/00; A61K 31/706; A61K 47/06; A61K 45/06; A61K 47/32; A61K 47/40

(52) U.S. Cl. CPC A61K 9/008; A61K 31/706; A61K 47/06; A61K 45/06; A61K 47/32; A61K 47/40

(57) ABSTRACT

A pharmaceutical composition is described. The composition comprises: (i) a drug component comprising at least one antiviral compound that is suitable for treating viruses that cause adverse effects in the lungs; and (ii) a propellant. The composition can be delivered to the lungs using a metered dose inhaler (MDI).

The present invention relates to pharmaceutical formulations that are designed to be delivered to the lungs using a suitable medical device. More particularly, the present invention relates to pharmaceutical compositions comprising a hydrofluorocarbon propellant, such as 1,1,1,2-tetrafluoroethane (HFA-134a) or 1,1-difluoroethane (HFA-152a), and an antiviral compound which is dissolved (optionally with the assistance of a polar excipient/co-solvent) or suspended in the propellant and to medical devices containing those compositions. The pharmaceutical compositions of the invention are particularly suited for delivery from a pressurised aerosol container using a metered dose inhaler (MDI).

A number of antiviral compounds are sold today for treating various viral infections. Such compounds are typically formulated as part of an injectable solution, usually an aqueous solution, and the formulations are administered intravenously.

One such antiviral compound that is currently available is remdesivir (2-ethylbutyl(2S)-2-[[[(2R,3S,4R,5R)-5-(4-aminopyrrolo[2,1-f][1,2,4]triazin-7-yl)-5-cyano-3,4-di hydroxyoxolan-2-yl]methoxy-phenoxyphosphoryl]amino]propanoate).

Remdesivir is the prodrug of an adenosine nucleotide analogue and is a broad spectrum antiviral compound that is thought to function by interfering with viral RNA-dependent RNA polymerase and as a result inhibit viral replication. Remdesivir has been investigated for use against a number of viral species including Ebola, SARS and MERS and has now been granted a license for use in humans where it has been found to reduce the recovery time from COVID-19 infection.

Remdesivir is currently administered intravenously as an aqueous solution. The intravenous delivery results in a widespread systemic exposure to the drug, which may be beneficial where the virus is widespread throughout the body or in an organ that cannot otherwise be treated directly. This distribution of drugs throughout the body requires large doses of the drug to be administered in order to reach an efficacious level throughout the mass of the patient. Unfortunately, the combination of large doses and widespread distribution can result in many undesirable systemic toxicity effects.

Furthermore, the stability of remdesivir in an aqueous solution is limited and it is usually stored as a lyophilised powder prior to being made up by addition of water. The solution may also contain a cyclodextrin to improve the solubility of the drug in water and an acid, typically HCl, to bring the pH of the solution to around 3-4 to improve hydrolytic stability.

With the recent pandemic caused by COVID-19, numerous research programmes are being conducted around the world to look for possible treatments as well as vaccinations.

As COVID-19 is a respiratory virus with many of the adverse effects taking place in the lung, it may be beneficial to provide a dose of antiviral drug directly to the lung. By dosing directly to the lung, the potential for systematic drug toxicity effects are reduced and the required dose to be administered is also reduced relative to intravenous delivery.

One well established mechanism for drug delivery to the lung is through a nebuliser. Here, an aqueous solution or suspension of the drug is atomised mechanically or by flow of air to produce a fine aerosol mist that is inhaled by the patient. The use of a nebuliser requires the drug to be soluble in water and to have adequate chemical, in particular hydrolytic, stability.

Another established method of drug delivery to the lung is the dry powder inhaler (DPI). Here, the drug, often co-mixed or suspended on a carrier particle such as lactose, is inhaled as a powder into the lung. The drug must be available in a particle size that is small enough to reach the deep lung (alveolar delivery). This usually requires the drug particles to have a size across their largest dimension in the range of from about 0.1 μm-10 μm. DPIs also require the patient to generate sufficient inspiratory airflow for the drug to be delivered deep into the lung in an adequate dosage. For patients with reduced lung functionality, for example as a result of a COVID-19 infection, this may prove difficult.

Another established system for delivering drugs to the lung is the metered dose inhaler (MDI). They are designed to deliver, on demand, a discrete and accurate amount of a drug to the respiratory tract of a patient as an aerosol spray using a pressurised liquefied propellant in which the drug is dissolved or suspended. The design and operation of MDIs is described in many standard textbooks and in the patent literature. They all comprise a pressurised container that holds the drug formulation, a nozzle and a valve assembly that is capable of dispensing a controlled quantity of the drug through the nozzle when it is activated. The nozzle and valve assembly are typically located in a housing that is equipped with a mouthpiece. The drug formulation comprises a liquified propellant, in which the drug is dissolved or suspended, and may contain other materials such as polar excipients to assist solubilisation, surfactants and preservatives.

Because the forces required to aerosolise the drug are provided by the propellant and do not rely as significantly on patient inspiratory flow, MDIs are more likely to be effective for a wider range of patients, particularly those with compromised lung functionality.

A useful MDI formulation is not without its challenges and requires a number of features including for suspension products: suspension physical stability (i.e. no or acceptably low levels of agglomeration, sedimentation and crystal growth), suspension chemical stability, and for solution products: adequate solubility in the propellant (with or without a co-solvent) and solution chemical stability.

In addition, in order for a prospective propellant to function satisfactorily in MDIs, it needs to have a number of properties. These include an appropriate boiling point and vapour pressure so that it can be liquefied in a closed container at room temperature but develop a high enough pressure when the MDI is activated to deliver the drug as an atomised formulation even at low ambient temperatures. Further, the propellant should be of low acute and chronic toxicity and have a high cardiac sensitisation threshold. It should have a high degree of chemical stability in contact with the drug, the container and the metallic and non-metallic components of the MDI device, and have a low propensity to extract low molecular weight substances from any elastomeric materials in the MDI device. The propellant should also be capable of maintaining the drug in a homogeneous solution or in a stable suspension for a sufficient time to permit reproducible delivery of the drug in use. When the drug is in suspension in the propellant, the density of the liquid propellant is desirably similar to that of the solid drug in order to avoid rapid sinking or floating of the drug particles in the liquid. Finally, the propellant should not present a significant flammability risk to the patient in use. In particular, it should form a non-flammable or low flammability mixture when mixed with air in the respiratory tract.

Dichlorodifluoromethane (R-12) possesses a suitable combination of properties and was for many years the most widely used MDI propellant, often blended with trichlorofluoromethane (R-11).

More recently, the hydrofluoroalkanes 1,1,1,2-tetrafluoroethane (HFA-134a) and 1,1,1,2,3,3,3-heptafluoropropane (HFA-227ea) have been introduced as MDI propellants. They can be used alone or blended together. Another useful hydrofluoroalkane propellant which is presently under evaluation is 1,1-difluoroethane (HFA-152a).

There is a need for a pharmaceutical composition comprising an antiviral medication that is suitable for treating viruses that cause adverse effects in the lungs, such as respiratory complications, that can be delivered using a MDI.

According to a first aspect of the present invention, there is provided a pharmaceutical composition, e.g. a pharmaceutical suspension or a pharmaceutical solution, said composition comprising:
(i) a drug component comprising at least one antiviral compound that is suitable for treating viruses that cause adverse effects in the lungs; and
(ii) a propellant.

The pharmaceutical composition of the present invention is suitable for delivery to the respiratory tract using a metered dose inhaler (MDI).

The propellant in the pharmaceutical composition of the present invention may be a chlorofluorocarbon or hydrochlorofluorocarbon propellant, such as R-11 or R-12, but in a preferred embodiment the propellant comprises one or more hydrofluorocarbons and, more particularly, one or more hydrofluoroalkanes. Suitable hydrofluoroalkanes for use in the pharmaceutical composition of the present invention include difluoromethane (HFA-32), 1,1,1,2-tetrafluoroethane (HFA-134a), 1,1,1,2,3,3,3-heptafluoropropane (HFA-227ea) and 1,1-difluoroethane (HFA-152a). HFA-134a, HFA-227ea and HFA-152a as well as their mixtures are preferred propellants, with HFA-134a, HFA-152a and mixtures thereof being particularly preferred and HFA-134a being especially preferred.

If the propellant component comprises one or more hydrofluoroalkanes, we do not exclude the possibility that it may also include other propellant compounds in addition to the hydrofluoroalkane(s). For example, the propellant component may additionally comprise one or more hydrocarbon propellant compounds, e.g. selected from propane, butane, isobutane and dimethyl ether.

Preferred propellants comprise at least 90 weight %, e.g. from 90 to 100 weight %, of one or more hydrofluoroalkanes. Preferably, the one or more hydrofluoroalkanes will constitute at least 95 weight %, e.g. from 95 to 100 weight %, and more preferably at least 99 weight %, e.g. from 99 to 100 weight %, of the propellant.

In an especially preferred embodiment, the propellant component consists of one or more hydrofluoroalkanes. By the term “consists of” we do not, of course, exclude the presence of minor amounts, e.g. up to a few hundred parts per million, of impurities that may be present following the process that is used to make the hydrofluoroalkane providing that they do not affect the suitability of the propellant in medical applications.

The amount of propellant component in the pharmaceutical composition of the invention will vary depending on the amounts of the drugs and other components in the pharmaceutical composition. Typically, the propellant component will comprise from 65.0 to 99.9 weight % of the total weight of the pharmaceutical composition. Preferably, the propellant component will comprise from 75.0 to 99.9 weight % and particularly from 83.0 to 99.9 weight % of the total weight of the pharmaceutical composition.

If the pharmaceutical composition only comprises the propellant component and the drug component, then the propellant component will typically comprise from 90.0 to 99.9 weight % of the total weight of the pharmaceutical composition with the drug component making up the remainder. Preferably, the propellant component will comprise from 95.0 to 99.9 weight % and particularly from 98.0 to 99.9 weight % of the total weight of the pharmaceutical composition with the drug component making up the remainder.

If the pharmaceutical composition comprises components in addition to the drug component and the propellant component, such as a polar excipient and/or a surfactant, then the propellant component will typically comprise from 65.0 to 99.0 weight % of the total weight of the pharmaceutical composition with the drug component and the additional components making up the remainder. Preferably, the propellant component will comprise from 75.0 to 98.5 weight % and particularly from 83.0 to 97.0 weight % of the total weight of the pharmaceutical composition with the drug component and the additional components making up the remainder.

The one or more antiviral compounds that are included in the pharmaceutical composition of the invention are those that are suitable for treating viruses that cause adverse effects in the lungs. Preferred antiviral compounds are prodrugs that are converted in the body to active metabolites that interfere with the action of viral RNA-dependent RNA polymerase. Particularly preferred antiviral compounds for use in the pharmaceutical composition of the invention are compounds that are converted into an adenosine nucleoside triphosphate analogue that interferes with the action of viral RNA-dependent RNA polymerase. The most preferred antiviral compound for use in the pharmaceutical composition of the invention is remdesivir.

The amount of the drug component in the pharmaceutical composition of the present invention will typically be in the range of from 0.1 to 10.0 weight % based on the total weight of the pharmaceutical composition. Preferably, the drug component will comprise from 0.1 to 5.0 weight % and particularly from 0.1 to 2.0 weight % of the total weight of the pharmaceutical composition.

The at least one antiviral compound may be dispersed or suspended in the propellant. The drug particles in such suspensions preferably have a diameter of less than 100 microns, e.g. less than 50 microns, and more preferably less than 10 microns, e.g. 5 microns or less.

However, in an preferred embodiment the pharmaceutical composition of the invention is a solution with the at least one antiviral compound dissolved in the propellant with the assistance of a polar excipient/co-solvent, such as ethanol, glycerol and/or water. Solution formulations have the potential to generate very fine aerosols with improved deep-lung penetration. This, in turn, may provide for reduced dose requirements as a result of more effective delivery to the lung and, therefore, lower undesirable systemic dosage.

The drug component may consist essentially of or consist entirely of the one or more antiviral compounds. By the term “consists essentially of”, we mean that at least 98 weight %, more preferably at least 99 weight % and especially at least 99.9 weight % of the drug component consists of the one or more antiviral compounds. Alternatively, the drug component may contain other respiratory drugs.

Suitable respiratory drugs that may be included with the one or more antiviral compounds include corticosteroids, long acting beta-2 agonists (LABA), long acting muscarinic antagonists and short acting muscarinic antagonists.

Suitable corticosteroids for use in the pharmaceutical composition of the invention include any of the corticosteroids that have been in use hitherto for treating respiratory disorders, such as asthma and chronic obstructive pulmonary diseases, and that can be delivered using a MDI. Accordingly, suitable corticosteroids include budesonide, mometasone, beclomethasone and fluticasone as well as their pharmaceutically acceptable derivatives, such as their pharmaceutically acceptable salts and esters.

Suitable long-acting beta-2-agonists (LABA) for use in the pharmaceutical composition of the invention include any of the long acting beta-2-agonists that have been in use hitherto for treating respiratory disorders, such as asthma and chronic obstructive pulmonary diseases, and that can be delivered using a MDI. Accordingly, suitable long acting beta-2-agonists include formoterol, arformoterol, bambuterol, clenbuterol, salmeterol, indacaterol, olodaterol and vilanterol as well as their pharmaceutically acceptable derivatives, such as their pharmaceutically acceptable salts and esters.

Suitable short-acting and long-acting muscarinic antagonists (LAMA) for use in the pharmaceutical composition of the invention include any of the short and long acting muscarinic antagonists that have been in use hitherto for treating respiratory disorders, such as chronic obstructive pulmonary diseases, and that can be delivered using a MDI. Accordingly, suitable short and long acting muscarinic antagonists include umeclidinium, ipratropium, tiotropium, aclidinium and the pharmaceutically acceptable derivatives thereof, especially the pharmaceutically acceptable salts thereof. Further suitable compounds include the pharmaceutically acceptable salts of glycopyrrolate (also known as glycopyrronium). Glycopyrrolate is a quaternary ammonium salt. Suitable pharmaceutically acceptable counter ions include, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. A preferred compound is the bromide salt of glycopyrrolate also known as glycopyrronium bromide.

In one embodiment, the pharmaceutical composition of the first aspect of the present invention consists essentially of and more preferably consists entirely of the two components (i) and (ii) listed above. By the term “consists essentially of”, we mean that at least 98 weight %, more preferably at least 99 weight % and especially at least 99.9 weight % of the pharmaceutical composition consists of the two listed components.

In a preferred embodiment of the invention, particularly when the pharmaceutical composition is a solution, the composition additionally includes a polar excipient. Polar excipients have been used previously in pharmaceutical compositions for treating respiratory disorders that are delivered using metered dose inhalers (MDIs). They are also referred to as solvents, co-solvents, carrier solvents and adjuvants. Their inclusion can serve to solubilise the drug and surfactant, if included, in the propellant and/or inhibit deposition of drug particles on the surfaces of the metered dose inhaler that are contacted by the pharmaceutical composition as it passes from the container in which it is stored to the nozzle outlet. They are also used as bulking agents in two-stage filling processes where the drug is mixed with a suitable polar excipient.

Suitable polar excipients are polar, oxygen-containing compounds such as ethanol, water, glycerol and polyalkylene glycols, e.g. polyethylene glycols, with ethanol and water and their mixtures being especially preferred.

If a polar excipient is used with the objective of forming a solution formulation, as is preferred, it will typically be present in an amount of from 0.5 to 25% by weight, preferably in an amount of from 1 to 20% by weight, e.g. in an amount of from 1 to 15% by weight, based on the total weight of the pharmaceutical composition. If a polar excipient is included for another purpose, e.g. as a means to dissolve another additive that is included in the pharmaceutical composition, such as a surfactant, or as a means to improve the suspension properties of the drug, then it will normally be included in smaller amounts, for example, in an amount of from 0.5 to 5% by weight based on the total weight of the pharmaceutical composition.

It will be appreciated from the discussion above that in a preferred embodiment the present invention provides a pharmaceutical composition, especially a solution formulation, that is adapted for delivery using a MDI, said composition comprising:
(i) a drug component comprising at least one antiviral compound that is suitable for treating viruses that cause adverse effects in the lungs, and especially remdesivir;
(ii) a propellant comprising at least one hydrofluoroalkane and especially a propellant comprising at least one hydrofluoroalkane selected from the group consisting of HFA-134a, HFA-227ea, HFA-152a and mixtures thereof; and
(iii) a polar excipient, particularly a polar excipient comprising at least one compound selected from ethanol, water and glycerol.

The pharmaceutical composition of the first aspect of the present invention may also include a surfactant component comprising at least one surfactant compound. The inclusion of a surfactant may be particularly useful when a suspension formulation is desired in order to avoid or ameliorate agglomeration and sedimentation issues with the suspended drug particles. Surfactant compounds of the type that have been in use hitherto in pharmaceutical formulations for MDIs may be used in the pharmaceutical compositions of the present invention. Preferred surfactants are selected from polyvinylpyrrolidone, polyethylene glycol surfactants, oleic acid and lecithin. By the term oleic acid, we are not necessarily referring to pure (9Z)-octadec-9-enoic acid. When sold for surfactant use in medical applications, oleic acid is typically a mixture of several fatty acids, with (9Z)-octadec-9-enoic acid being the predominant fatty acid, e.g. present in an amount of at least 65 weight % based on the total weight of the surfactant. Particularly preferred surfactants are polyvinylpyrrolidone, oleic acid and lecithin, especially oleic acid and lecithin.

If a surfactant component is used, it will typically be present in an amount of from 0.1 to 2.5% by weight, preferably in an amount of from 0.2 to 1.5% by weight based on the total weight of the pharmaceutical composition.

Accordingly, the present invention also provides a pharmaceutical composition, especially a suspension formulation, that is adapted for delivery using a MDI, said composition comprising:
(i) a drug component comprising at least one antiviral compound that is suitable for treating viruses that cause adverse effects in the lungs, and particularly remdesivir;
(ii) a propellant comprising at least one hydrofluoroalkane and particularly a propellant comprising at least one hydrofluoroalkane selected from the group consisting of HFA-134a, HFA-227ea, HFA-152a and mixtures thereof; and
(iii) a surfactant component comprising at least one surfactant compound, particularly at least one surfactant compound selected from polyvinylpyrrolidone, oleic acid and lecithin.

In another embodiment, the present invention provides a pharmaceutical composition and preferably a solution that is adapted for delivery using a MDI, said composition comprising:
(i) a drug component comprising at least one antiviral compound that is suitable for treating viruses that cause adverse effects in the lungs, and particularly remdesivir;
(ii) a propellant comprising at least one hydrofluoroalkane and particularly a propellant comprising at least one hydrofluoroalkane selected from the group consisting of HFA-134a, HFA-227ea, HFA-152a and mixtures thereof;
(iii) a polar excipient, particularly a polar excipient comprising at least one compound selected from ethanol, water and glycerol; and
(iv) a surfactant component comprising at least one surfactant compound particularly at least one surfactant compound selected from polyvinylpyrrolidone, oleic acid and lecithin.

The pharmaceutical composition of the present invention may also include an acid stabiliser, such as an organic or inorganic (mineral) acid. Acid stabilisers may be particularly beneficial in solution formulations that contain water in order to improve hydrolytic stability. Preferred acid stabilisers are citric acid and hydrochloric acid.

Aqueous pharmaceutical solutions of the present invention may also contain an additive to improve the solubility of the antiviral drug and especially remdesivir in water. Suitable additives for enhancing solubility are oligosaccharides, such as the cyclodextrins. especially beta-cyclodextrin.

Suspension formulations of the present invention may also comprise a co-suspension agent. Suitable co-suspension agents are porous particles, such as perforated microstructures. Suitable perforated microstructures are described in WO 99/16422. In a preferred embodiment, the co-suspension agent comprises perforated microstructures made by spray drying phospholipid-based agents, such as calcium salts of distearoyl phosphatidylcholine. Other suitable materials for manufacturing perforated microstructures include lactose and amino-acids, such as leucine.

The pharmaceutical compositions of the invention may also comprise one or more other additives of the type that are conventionally used in drug formulations for pressurised MDIs, such as valve lubricants. Where other additives are included in the pharmaceutical compositions, they are normally used in amounts that are conventional in the art.

The preferred pharmaceutical solutions of the present invention are the following:

A pharmaceutical solution for delivery using a MDI, said solution comprising:
(i) a drug component comprising remdesivir;
(ii) a propellant comprising at least one hydrofluoroalkane selected from the group consisting of HFA-134a, HFA-227ea, HFA-152a and mixtures thereof; and
(iii) a polar excipient comprising ethanol, water or an ethanol/water mixture.

A pharmaceutical solution for delivery using a MDI, said solution comprising:
(i) a drug component comprising remdesivir;
(ii) a propellant comprising at least one hydrofluoroalkane selected from the group consisting of HFA-134a, HFA-227ea, HFA-152a and mixtures thereof;
(iii) a polar excipient comprising ethanol, water or an ethanol/water mixture; and
(iv) a surfactant component comprising at least one surfactant compound selected from oleic acid and lecithin.

The drug component in the preferred pharmaceutical solutions described above is dissolved in the mixture of the propellant and polar excipient.

In the first of the above preferred pharmaceutical solutions, the drug component preferably comprises from 0.1 to 10.0 weight %, the propellant from 65.0 to 99.0 weight % and the polar excipient from 0.5 to 25.0 weight % of the total weight of the pharmaceutical solution.

In the second of the above preferred pharmaceutical solutions, the drug component preferably comprises from 0.1 to 10.0 weight %, the propellant from 65.0 to 99.0 weight %, the polar excipient from 0.5 to 25.0 weight % and the surfactant component from 0.1 to 2.5 weight % of the total weight of the pharmaceutical solution.

In one embodiment of the preferred pharmaceutical solutions described above, the propellant is HFA-134a.

In another embodiment of the preferred pharmaceutical solutions described above, the propellant is HFA-152a.

In a further embodiment of the preferred pharmaceutical solutions described above, the propellant is HFA-134a and the polar excipient is ethanol.

In another embodiment of the preferred pharmaceutical solutions described above, the propellant is HFA-152a and the polar excipient is ethanol.

In a further embodiment of the preferred pharmaceutical solutions described above, the propellant is HFA-134a and the polar excipient is a mixture of water and ethanol. These solutions may also comprise an acid stabiliser selected from citric acid and hydrochloric acid.

In another embodiment of the preferred pharmaceutical solutions described above, the propellant is HFA-152a and the polar excipient is a mixture of water and ethanol. These solutions may also comprise an acid stabiliser selected from citric acid and hydrochloric acid.

The preferred pharmaceutical suspensions of the present invention are the following:

A pharmaceutical suspension for delivery using a MDI, said suspension comprising:
(i) a drug component comprising remdesivir; and
(ii) a propellant comprising at least one hydrofluoroalkane selected from the group consisting of HFA-134a, HFA-227ea, HFA-152a and mixtures thereof.

A pharmaceutical suspension for delivery using a MDI, said suspension comprising:
(i) a drug component comprising remdesivir;
(ii) a propellant comprising at least one hydrofluoroalkane selected from the group consisting of HFA-134a, HFA-227ea, HFA-152a and mixtures thereof; and
(iii) a polar excipient comprising ethanol.

A pharmaceutical suspension for delivery using a MDI, said suspension comprising:
(i) a drug component comprising remdesivir;
(ii) a propellant comprising at least one hydrofluoroalkane selected from the group consisting of HFA-134a, HFA-227ea, HFA-152a and mixtures thereof;
(iii) a polar excipient comprising ethanol; and
(iv) a surfactant component comprising at least one surfactant compound selected from polyvinylpyrrolidone, oleic acid and lecithin.

The drug component in the preferred pharmaceutical suspensions described above is suspended in the mixture of the propellant and polar excipient.

In the first of the above preferred pharmaceutical suspensions, the drug component preferably comprises from 0.1 to 10.0 weight % and the propellant from 90.0 to 99.9 weight % of the total weight of the pharmaceutical suspension.

In the second of the above preferred pharmaceutical suspensions, the drug component preferably comprises from 0.1 to 10.0 weight %, the propellant from 85.0 to 99.0 weight % and the polar excipient from 0.5 to 5.0 weight % of the total weight of the pharmaceutical suspension.

In the third of the above preferred pharmaceutical suspensions, the drug component preferably comprises from 0.1 to 10 weight %, the propellant from 83.0 to 99.0 weight %, the polar excipient from 0.5 to 5.0 weight % and the surfactant component from 0.1 to 2.5 weight % of the total weight of the pharmaceutical suspension.

In one embodiment of the preferred pharmaceutical suspensions described above, the propellant is HFA-134a.

In another embodiment of the preferred pharmaceutical suspensions described above, the propellant is HFA-152a.

The pharmaceutical compositions of the invention find particular utility in the delivery of the antiviral compound and especially remdesivir from a pressurised aerosol container, e.g. using a metered dose inhaler (MDI). For this application, the pharmaceutical compositions are contained in the pressurised aerosol container which is used in association with the MDI. In a particularly preferred embodiment, the pressurised container is a coated aluminium can or an uncoated aluminium can, especially the latter. When so stored, the pharmaceutical compositions are normally a liquid and the propellant functions to deliver the drug as a fine aerosol spray.

Accordingly, a second aspect of the present invention provides a pressurised container holding a pharmaceutical composition as defined herein. In a third aspect, the present invention provides a metered dose inhaler having a pressurised container holding a pharmaceutical composition as defined herein.

The metered dose inhaler typically comprises a nozzle and valve assembly that is crimped to a container holding the pharmaceutical composition to be dispensed. An elastomeric gasket is used to provide a seal between the container and the nozzle/valve assembly. Preferred elastomeric gasket materials are EPDM, chlorobutyl, bromobutyl and cycloolefin copolymer rubbers.

The pharmaceutical compositions of the present invention are for use in medicine for treating a patient suffering from a viral infection, especially a viral infection that attacks the lungs causing concomitant respiratory problems.

Accordingly, the present invention also provides a method for treating a patient suffering from a viral infection which comprises administering to the patient a therapeutically effective amount of a pharmaceutical composition as defined herein. The pharmaceutical composition is preferably delivered to the patient using a MDI. The present invention is directed in particular to the treatment of Covid-19.

The present invention also provides a pharmaceutical composition as defined herein for use in treating a viral infection that cause adverse effects in the lungs.

Also provided is the use of a pharmaceutical composition as defined herein for manufacturing a medicament for treating a viral infection that cause adverse effects in the lungs.

The pharmaceutical compositions of the invention can be prepared and the MDI devices filled using techniques that are standard in the art, such as pressure filling and cold filling. For example, the pharmaceutical compositions can be prepared by a simple blending operation in which the at least one antiviral compound, the propellant and any optional additives, such as another respiratory drug, a polar excipient and/or a surfactant component, are mixed together in the required proportions in a suitable mixing vessel. Mixing can be promoted by stirring as is conventional in the art. Conveniently, the propellant is liquefied to aid mixing. If the pharmaceutical composition is made in a separate mixing vessel, it can then be transferred to pressurised containers for storage, such as pressurised containers that are used as part of medication delivery devices and especially MDIs.

The pharmaceutical compositions of the invention can also be prepared within the confines of a pressurised container, such as an aerosol canister or vial, from which the compositions are ultimately released as an aerosol spray using a medication delivery device, such as a MDI. In this method, a weighed amount of the at least one antiviral compound, and optionally any further respiratory drugs that are to be included, is introduced into the open container. A valve is then crimped onto the container and the liquid propellant introduced through the valve into the container under pressure, optionally after first evacuating the container through the valve. The polar excipient and surfactant component, if included, and any other additives can be mixed with the drug(s) or, alternatively, introduced into the container after the valve has been fitted, either alone or as a premix with the propellant. The whole mixture can then be treated to mix the components together, e.g. by vigorous shaking or using an ultrasonic bath.

Suitable containers may be made of plastics, metal, e.g. aluminium, or glass. Preferred containers are made of metal, especially aluminium which may be coated or uncoated. Uncoated aluminium containers are especially preferred.

The container may be filled with enough of the pharmaceutical composition to provide for a plurality of dosages. The pressurized aerosol canisters that are used in MDIs typically contain 50 to 150 individual dosages.

The present invention is now illustrated but not limited by the following examples

### EXAMPLE 1

MDIs containing solution formulations of remdesivir may be prepared in a mixing vessel by adding a weighed amount of remdesivir and adding the requisite quantity of water. If desired, beta-cyclodextrin is also added at this stage. The mixture is stirred until the remdesivir has dissolved. If desired, HCl(aq) is added to bring the pH of the resulting solution to between pH3 and pH4. The aqueous solution is then mixed with ethanol and the resulting solution having a remdesivir concentration of at least 5 mg/mL is transferred in aliquots to an open MDI can (H&T Presspart 19 mL can size) and crimped closed with the valve (Aptar DF30). The propellant (HFA-134a, HFA-152a) is pressure filled into the can. Suitable final compositions in the can are recorded in Table 1 below.

TABLE 1

| | 1A | 1B | 1C | 1D |
|---|---|---|---|---|
| Remdesivir(mg) | 100 | 50 | 100 | 50 |
| Water(g) | 1 | 1 | 0.5 | 0.5 |
| Ethanol(g) | 2 | 1 | 3 | 2 |
| Propellant | q.s. | q.s. | q.s. | q.s. |

### EXAMPLE 2

MDIs containing solution formulations of remdesivir may be prepared in a mixing vessel by adding a weighed amount of remdesivir and adding the requisite quantity of ethanol. The mixture is stirred until the remdesivir has dissolved. The solution is transferred in aliquots to an open MDI can (H&T Presspart 19 mL can size) and crimped closed with the valve (Aptar DF30). The propellant (HFA-134a, HFA-152a) is pressure filled into the can. Suitable final compositions in the can are recorded in Table 2 below.

TABLE 2

| | 2A | 2B | 2C | 2D |
|---|---|---|---|---|
| Remdesivir(mg) | 100 | 50 | 100 | 50 |
| Ethanol(g) | 2 | 1 | 4 | 2 |
| Propellant | q.s. | q.s. | q.s. | q.s. |

### EXAMPLE 3

MDIs containing suspension formulations of remdesivir may be prepared in a mixing vessel capable of containing the propellant by adding a weighed amount of remdesivir and if desired, the requisite quantity of ethanol and either PVP or oleic acid surfactant. The propellant is added and the mixture stirred before it is pressure-transferred in aliquots to a pre-crimped MDI can (H&T Presspart 19 mL can size) with the Aptar DF30 valve. Suitable final compositions in the can are recorded in Table 3 below.

TABLE 3

| | 3A | 3B | 3C | 3D |
|---|---|---|---|---|
| Remdesivir(mg) | 100 | 50 | 100 | 50 |
| Ethanol(g) | 0 | 0 | 0.1 | 0.1 |
| PVP(mg) | 0.01 | 0 | 0 | 0.01 |
| Oleic acid(mg) | 0 | 0.01 | 0.01 | 0 |
| Propellant | q.s. | q.s. | q.s. | q.s. |

1. A pharmaceutical composition comprising:
(i) a drug component comprising at least one antiviral compound that is suitable for treating viruses that cause adverse effects in the lungs; and
(ii) a propellant.

2. (canceled)

3. The pharmaceutical composition of claim 1, wherein the at least one antiviral compound is a prodrug of an adenosine nucleoside analogue.

4. The pharmaceutical composition of claim 1, wherein the at least one antiviral compound includes remdesivir.

5. (canceled)

6. The pharmaceutical composition of claim 1, wherein the drug component additionally comprises at least one further respiratory drug selected from the corticosteroids, the long acting beta-2 agonists (LABA), the long acting muscarinic antagonists and the short acting muscarinic antagonists.

7. The pharmaceutical composition of claim 1, wherein the propellant component comprises at least one hydrofluoroalkane.

8. The pharmaceutical composition of claim 7, wherein the at least one hydrofluoroalkane is selected from the group consisting 1,1,1,2-tetrafluoroethane (HFA-134a), 1,1,1,2,3,3,3-heptafluoropropane (HFA-227ea), 1,1-difluoroethane (HFA-152a) and mixtures thereof.

9-10. (canceled)

11. The pharmaceutical composition of claim 1, further comprising a polar excipient.

12. The pharmaceutical composition of claim 11, wherein the polar excipient is selected from the group consisting of water, ethanol, glycerol and their mixtures.

13. (canceled)

14. The pharmaceutical composition of claim 1, further comprising an acid stabiliser.

15. The pharmaceutical composition of claim 1 in the form of a suspension.

16. The pharmaceutical composition of claim 15 further comprising a co-suspension agent.

17. The pharmaceutical composition of claim 11 in the form of a solution.

18. The pharmaceutical composition of claim 17 further comprising an additive to increase the solubility of the antiviral compound in the mixture of the propellant and polar excipient.

19. The pharmaceutical composition of claim 1, further comprising a surfactant component comprising at least one surfactant compound.

20. A pharmaceutical solution for delivery using a metered dose inhaler (MDI) comprising:
(i) a drug component comprising remdesivir;
(ii) a propellant comprising at least one hydrofluoroalkane selected from the group consisting of 1,1,1,2-tetrafluoroethane (HFA-134a), 1,1,1,2,3,3,3-heptafluoropropane (HFA-227ea), 1,1-difluoroethane (HFA-152a) and mixtures thereof; and
(iii) a polar excipient comprising ethanol, water or an ethanol/water mixture.

21. (canceled)

22. The pharmaceutical solution of claim 20, further comprising (iv) a surfactant component comprising at least one surfactant compound selected from oleic acid and lecithin.

23. A pharmaceutical suspension for delivery using a MDI comprising:
(i) a drug component comprising remdesivir; and
(ii) a propellant comprising at least one hydrofluoroalkane selected from the group consisting of 1,1,1,2-tetrafluoroethane (HFA-134a), 1,1,1,2,3,3,3-heptafluoropropane (HFA-227ea), 1,1-difluoroethane (HFA-152a) and mixtures thereof.

24. The pharmaceutical suspension of claim 23 further comprising (iii) ethanol.

25. (canceled)

26. The pharmaceutical suspension of claim 23, further comprising (iv) a surfactant component comprising at least one surfactant compound selected from polyvinylpyrrolidone, oleic acid and lecithin.

27-35. (canceled)

36. A sealed and pressurized aerosol container for use with a metered dose inhaler (MDI) that contains a pharmaceutical composition of claim 1.

37. A metered dose inhaler (MDI) fitted with a sealed and pressurized aerosol container of claim 36.

38. A method for treating a patient suffering from a viral infection that causes adverse effects in the lungs, said method comprising
administering to the patient a therapeutically effective amount of a pharmaceutical composition of claim 1.

39. The method of claim 38, wherein the pharmaceutical composition is delivered to the patient using a metered dose inhaler (MDI).

40. The method of claim 38, wherein the viral infection that causes adverse effects in the lungs is Covid-19.

41-42. (canceled)
